SEQUENCE LISTING (8 sequences)

SEQ ID NO: 1 - protein, 117 aa, synthetic construct
  REGION 1..117: note = humanized antibody sequence
EVQLEESGGGLVQPGGSLRLSCAASGFDFSSNTMCWVRQAPGKGLEWVACIYAGSSGSTYYASWAKGRFTISKDISKTTLYLQMNSLRAEDTATYFCARGLSRFSLWGQGTLVTVSS

SEQ ID NO: 2 - protein, 111 aa, synthetic construct
  REGION 1..111: note = humanized antibody sequence
DIVMTQSPSSLSASVGDRVTITCQASQSISNYLAWYQQKPGQAPKLLIYDASKLASGVPSRFSGSGSGTDFTLTISSLQPEDFATYYCQGGDYYGSSYVVAFGGGTKVVIK

SEQ ID NO: 3 - protein, 5 aa, synthetic construct
  REGION 1..5: note = humanized antibody sequences
SNTMC

SEQ ID NO: 4 - protein, 18 aa, synthetic construct
  REGION 1..18: note = humanized antibody sequence
CIYAGSSGSTYYASWAKG

SEQ ID NO: 5 - protein, 7 aa, synthetic construct
  REGION 1..7: note = humanized antibody sequence
GLSRFSL

SEQ ID NO: 6 - protein, 11 aa, synthetic construct
  REGION 1..11: note = humanized antibody sequence
QASQSISNYLA

SEQ ID NO: 7 - protein, 7 aa, synthetic construct
  REGION 1..7: note = humanized antibody sequence
DASKLAS

SEQ ID NO: 8 - protein, 13 aa, synthetic construct
  REGION 1..13: note = humanized antibody sequence
QGGDYYGSSYVVA

(19) United States
(12) Patent Application Publication
(10) Pub. No.: US 2024/0392026 A1
(43) Pub. Date: Nov. 28, 2024

(54) AGONISTIC CD40 ANTIBODIES AS IMMUNE STIMULATORY AGENTS

(71) Applicant: MAB Discovery GmbH, Polling (DE)

(72) Inventor: Stephan FISCHER, Polling (DE)

(73) Assignee: MAB Discovery GmbH, Polling (DE)

(21) Appl. No.: 18/696,925

(22) PCT Filed: Sep. 30, 2022
(86) PCT No.: PCT/EP2022/077270

§ 371 Date: Mar. 28, 2024

(30) Foreign Application Priority Data
Oct. 1, 2021 (EP) 21200496.4

(51) Int. Cl. C07K 16/28; A61K 39/00; A61K 39/395; A61K 45/06; A61P 37/04

(52) U.S. Cl. CPC C07K 16/2878; A61K 39/3955; A61K 45/06; A61P 37/04; A61K 2039/505; C07K 2317/24; C07K 2317/75

(57) ABSTRACT

The present invention relates to humanized monoclonal agonistic antibodies or antigen-binding fragments thereof that specifically bind to human CD40 receptor and are capable of inducing CD40 signaling independent of Fey mediated CD40 receptor crosslinking for use as immune stimulatory agents.

### FIELD OF INVENTION

The present invention relates to humanized monoclonal agonistic antibodies or antigen-binding fragments thereof that specifically bind to human CD40 receptor and are capable of inducing CD40 signaling independent of Fcγ mediated CD40 receptor crosslinking for use as immune stimulatory agents.

### BACKGROUND

Recent success in cancer immunotherapy has revived the hypothesis that the immune system can control many if not most cancers, in some cases producing durable responses in a way not seen with many small-molecule drugs. Agonistic CD40 monoclonal antibodies (mAb) offer a new therapeutic option, which has the potential to generate anticancer immunity by various mechanisms.

CD40 is a cell-surface molecule and a member of the tumor necrosis factor (TNF) receptor superfamily. It is expressed broadly on antigen-presenting cells (APC) such as dendritic cells, B cells, and monocytes as well as many nonimmune cells and in a range of tumors.

The natural ligand for CD40 is CD154, which is expressed primarily on the surface of activated T lymphocytes and provides a major component of T-cell “help” for immune responses: Signaling via CD40 on APC mediates, in large part, the capacity of helper T cells to license APC. Ligation of CD40 on DC, for example, induces increased surface expression of costimulatory and MHC molecules, production of proinflammatory cytokines, and enhanced T-cell triggering. CD40 ligation on resting B cells increases antigen-presenting function and proliferation.

The consequences of CD40 signaling are multifaceted and depend on the type of cell expressing CD40 and the microenvironment in which the CD40 signal is provided. Like some other members of the TNF receptor family, CD40 signaling is mediated by adapter molecules rather than by inherent signal transduction activity of the CD40 cytoplasmic tail. Downstream kinases are activated when the receptor is assembled, a multicomponent signaling complex translocates from CD40 to the cytosol and several well-characterized signal transduction pathways are activated.

Antagonizing human CD40 antibodies are known in the prior art. Most of the clinically tested therapeutic anti-CD40 antibodies work via FcγR crosslinking, although data have indicated a correlation between toxicity and FcγR crosslinking. Therefore, silent Fc variants, showing a reduced Fcγ mediated CD40 receptor cross-linking have been developed. Respective mutations of the human IgG1 FC region are described in for example US 2018/0118843.

In recently designed immunomodulatory approaches, CD40-targeting agonist monoclonal antibodies (mAbs) are used to enhance the ability of the immune system to recognize and destroy cancer cells. Respective pre-clinical studies have shown that agonistic CD40 mAb can activate APC and promote antitumor T-cell responses and to foster cytotoxic myeloid cells with the potential to control cancer in the absence of T-cell immunity. Thus, agonistic CD40 mAb are fundamentally different from mAb that accomplish immune activation by blocking negative check-point molecules such as CTLA-4 or PD-1.

WO 2019/057792 describes humanized monoclonal agonistic antibodies or antigen-binding fragments thereof that specifically bind to human CD40 receptor and are capable of inducing CD40 signaling independent of Fcγ mediated CD40 receptor crosslinking.

Thus, agonistic CD40 mAbs represent a promising strategy for novel immune therapies, in particular for cancer therapeutics. However, concerns have been raised in respect to their potential cytotoxic side-effects. Agonistic monoclonal CD40 antibodies stand in prospect of triggering cytokine release syndromes, autoimmune reactions, thromboembolic syndromes (due to the expression of CD40 by platelets and endothelial cells), hyper immune stimulation leading to activation-induced cell death or tolerance, and tumor angiogenesis. These effects may cause untoward toxicity or the promotion of tumor growth. Mechanistically, the ability of agonistic CD40 and other TNF receptor family targeting antibodies to interact with Fcγ receptors has been linked to the occurrence of toxicities in animal studies (Li & Ravetch 2012, Xu et al. 2003, Byrne et al. 2016).

Thus, there is a need to provide an effective immunotherapy with minimal or no toxic side effects. In the present application, the immunomodulatory potential of agonistic CD40 antibodies was evaluated. It turned out that certain antibodies have a surprisingly large therapeutic window in which they elicit an immune stimulatory effect without causing excessive toxic effects.

### SUMMARY OF INVENTION

The present invention provides monoclonal antibodies or antigen-binding fragments thereof that specifically bind to human CD40 receptor and induce CD40 signaling independent of Fcγ mediated CD40 receptor crosslinking for use as immune stimulatory agents. According to the present invention, the antibodies or antibody fragments are provided for administration to a patient in a dose of about 0.1 to 1 mg per kg body weight of the patient. In this range, they are capable to elicit a strong immune stimulatory effect while toxic side effects, if any, are negligible. The present invention also provides pharmaceutical compositions comprising said antibodies adapted for use in the treatment of a condition or disease, in which the stimulation of the immune system is desired, e.g. in the prevention or treatment of infectious diseases and for treating patients suffering from cancer.

### Definitions

The term “antibody” encompasses the various forms of antibody structures including, but not being limited to, whole antibodies and antibody fragments as long as it shows the properties according to the invention.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F (ab′) 2; diabodies; linear antibodies; single-chain antibody molecules (e.g. scFv); and multi-specific antibodies formed from antibody fragments.

The terms “monoclonal antibody” or “monoclonal antibody composition” as used herein refer to a preparation of antibody molecules of a single amino acid composition.

The term “humanized antibody” or “humanized version of an antibody” refers to antibodies for which both heavy and light chains are humanized as a result of antibody engineering. A humanized chain is typically a chain in which the V-region amino acid sequence has been changed so that, analyzed as a whole, is closer in homology to a human germline sequence than to the germline sequence of the species of origin. Humanization assessment is based on the resulting amino acid sequence and not on the methodology per se.

The terms “specifically binding, against target, or anti-target antibody”, as used herein, refer to binding of the antibody to the respective antigen (target) or antigen-expressing cell, measured by ELISA, wherein said ELISA preferably comprises coating the respective antigen to a solid support, adding said antibody under conditions to allow the formation of an immune complex with the respective antigen or protein, detecting said immune complex by measuring the Optical Density values (OD) using a secondary antibody binding to an antibody according to the invention and using a peroxidase-mediated color development.

The term “antigen” according to the invention refers to the antigen used for immunization or a protein comprising said antigen as part of its protein sequence. For example, for immunization a fragment of the extracellular domain of a protein (e.g. the first 20 amino acids) can be used and for detection/assay and the like the extracellular domain of the protein or the full-length protein can be used.

The term “specifically binding” or “specifically recognized” herein means that an antibody exhibits appreciable affinity for an antigen and, preferably, does not exhibit significant cross-reactivity.

An antibody that “does not exhibit significant cross-reactivity” is one that will not appreciably bind to an undesirable other protein. Specific binding can be determined according to any art-recognized means for determining such binding, e.g. by competitive binding assays such as ELISA.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The “variable region (or domain) of an antibody according to the invention” (variable region of a light chain (VL), variable region of a heavy chain (VH)) as used herein denotes each of the pair of light and heavy chain regions which are involved directly in binding the antibody to the antigen. The variable light and heavy chain regions have the same general structure and each region comprises four framework (FR) regions whose sequences are widely conserved, connected by three complementary determining regions, CDRs.

The term “antigen-binding portion of an antibody” when used herein refers to the amino acid residues of an antibody which are responsible for antigen binding. The antigen-binding portion of an antibody comprises preferably amino acid residues from the “complementary determining regions” or “CDRs”. The CDR sequences are defined according to Kabat et al, Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable region. For example, a heavy chain variable region may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The “constant domains (constant parts)” are not involved directly in binding of an antibody to an antigen, but exhibit e.g. also effector functions. The heavy chain constant region gene fragment that corresponds to human IgG1 is called γ1 chain. The heavy chain constant region gene fragment that correspond to human IgG3 is called γ3 chain. Human constant γ heavy chains are described in detail by Kabat, E. A. et al., Sequences of Proteins of Immunological Interest, 5th ed., Public Health Service, National Institutes of Health, Bethesda, MD. (1991), and by Brueggemann, M., et al., J. Exp. Med. 166 (1987) 1351-1361; Love, T. W., et al., Methods Enzymol. 178 (1989) 515-527.

The term “Fc region” herein is used to define a C-terminal region of an immunoglobulin heavy chain that contains at least a portion of the constant region. The term includes native sequence Fc regions and variant Fc regions.

Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

A “variant Fc region” comprises an amino acid sequence which differs from that of a “native” or “wildtype” sequence Fc region by virtue of at least one “amino acid modification” as herein defined.

The term “Fc-variant” as used herein refers to a polypeptide comprising a modification in the Fc domain. The modification can be an addition, deletion, or substitution. Substitutions can include naturally occurring amino acids and non-naturally occurring amino acids. Variants may comprise non-natural amino acids.

The term “Fc region-containing polypeptide” refers to a polypeptide, such as an antibody, which comprises an Fc region.

The terms “Fc receptor” or “FcR” are used to describe a receptor that binds to the Fc region of an antibody. A FcR which binds an IgG antibody (a gamma receptor) includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain, (see review in Daeron, M., Annu. Rev. Immunol. 15 (1997) 203-234). FcRs are reviewed in Ravetch, and Kinet, Annu. Rev. Immunol 9 (1991) 457-492; Capel, et al., Immunomethods 4 (1994) 25-34; and de Haas, et al., J. Lab. Clin. Med. 126 (1995) 330-41. Other FcRs, including those to be identified in the future, are encompassed by the term “FcR” herein. The term also includes the neonatal receptor, FcRn, which is responsible for the transfer of maternal IgGs to the fetus (Guyer, et al., J. Immunol. 117 (1976) 587 and Kim, et al., J. Immunol. 24 (1994) 249).

By “IgG Fc ligand” as used herein means a molecule, preferably a polypeptide, from any organism that binds to the Fc region of an IgG antibody to form an Fc/Fc ligand complex. Fc ligands include but are not limited to FcyRs, FcRn, Clq, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis, et al., Immunological Reviews 190 (2002) 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

“Fc gamma receptor”, “FcyR” or “FcgammaR” as used herein means any member of the family of proteins that bind the IgG antibody Fc region and is encoded by an FcyR gene. In humans, this family includes but is not limited to FcyRI (CD64), including isoforms FcyRIA, FcyRIB, and FcyRIC; FcyRII (CD32), including isoforms FcyRIIA (including allotypes H131 and R131), FcyRIIB (including FcyRIIB-I and FcyRIIB-2), and FcyRIIc; and FcyRIII (CD 16), including isoforms FcyRIIIA (including allotypes V158 and F158) and FcyRIIIb (including allotypes FcyRIIB-NAI and FcyRIIB-NA2) (Jefferis, et al., Immunol Lett 82 (2002)), as well as any undiscovered human FcyRs or FcyR isoforms or allotypes. An FcyR may be from any organism, including but not limited to humans, mice, rats, rabbits, and monkeys. Mouse FcyRs include but are not limited to FcyRI (CD64), FcyRII (CD32), FcyRIII (CD 16), and FCYRIII-2 (CD 16-2), as well as any undiscovered mouse FcγRs or FcγR isoforms or allotypes.

“FcRn” or “neonatal Fc Receptor” as used herein refers to a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

“Antibody-dependent cell-mediated cytotoxicity” and “ADCC” refer to a cell mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. FcR expression on hematopoietic cells is summarized in Table 3 on page 464 of Ravetch, and Kinet, Annu. Rev. Immunol 9 (1991) 457-492.

The terms “antibody-dependent cellular phagocytosis” and “ADCP” refer to a process by which antibody-coated cells are internalized, either in whole or in part, by phagocytic immune cells (e.g., macrophages, neutrophils and dendritic cells) that bind to an immunoglobulin Fc region.

The term “antibody effector function(s)” or “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An “effective amount” of an agent, e.g., a pharmaceutical formulation, refers to an amount effective, at dosages and for periods of time necessary, to achieve the desired therapeutic or prophylactic result.

The term “infectious disease” as used herein refers to any disease that is caused by an infectious organism. Infectious organisms may comprise viruses, (e.g. RNA viruses, DNA viruses, human immunodeficiency virus (HIV), hepatitis A, B, and C virus, herpes simplex virus (HSV), cytomegalovirus (CMV), Epstein-Barr virus (EBV), human papilloma virus (HPV)), parasites (e.g. protozoan and metazoan pathogens such as plasmodia species, Leishmania species, Schistosoma species, Trypanosoma species), bacteria (e.g. Mycobacteria, in particular, M. tuberculosis, Salmonella, Streptococci, E. coli, Staphylococci), funghi (e.g. Candida species, Aspergillus species), Pneumocystis carinii, and prions.

The term “cancer” as used herein may be, for example, lung cancer, non-small cell lung (NSCL) cancer, bronchioloalveolar cell lung cancer, bone cancer, pancreatic cancer, advanced pancreatic carcinoma skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers.

As used herein, the term “immunotherapy” refers to a treatment, for example, a therapeutic or prophylactic treatment, of a disease or disorder intended to and/or producing an immune response (e.g., an active or passive immune response).

The term “immunotherapeutic agent” refers to an agent that comprises or consists of one or more immunogens, immunoglobulins, antibodies, or antibody fragments, or combinations thereof, for use in immunotherapy. Accordingly, as used herein, the term “immunotherapeutic agent” also includes nucleic acids encoding immunogens, immunoglobulins, antibodies, or antibody fragments. Such nucleic acids can be DNA or RNA. A nucleic acid segment encoding an immunogen is typically linked to regulatory elements, such as a promoter and enhancer that allow expression of the DNA segment in the intended target cells of a subject or patient.

An “immunogenic agent” or “immunogen” is capable of inducing an immunological response against itself on administration to a patient, optionally in conjunction with an adjuvant. An “immunogenic composition” is one that comprises an immunogenic agent.

The term “adjuvant” refers to a compound that when administered in conjunction with an immunogen augments the immune response to the immunogen, but when administered alone does not generate an immune response. Adjuvants can augment an immune response by several mechanisms including lymphocyte recruitment, stimulation of B and/or T cells, and stimulation of macrophages.

### DETAILED DESCRIPTION OF THE INVENTION

The present invention serves the need for providing agonistic CD40 antibodies or antigen-binding fragments thereof as immune stimulatory agents in medical treatment. According to the present invention, the antibodies or antibody fragments are provided for administration to a patient in a dose of about 0.1 to 1 mg per kg body weight of the patient. In this range, the specific antibodies described herein are capable to provide the desired signaling potency and clinical effectiveness without eliciting significant cellular toxicity.

It was found that one of the agonistic CD40 antibodies of the group of antibodies generally described in WO2019/057792 has a particularly large therapeutic window. It is already effective in very small doses as low as 0.1 mg per body weight of a patient and major toxic effects only occur at doses more than 10 times higher. The antibody or antigen-binding fragment thereof for use according to the present invention comprises a VH region comprising a CDR1H region of SEQ ID NO: 3, a CDR2H region of SEQ ID NO: 4 and a CDR3H region of SEQ ID NO: 5, and a VL region comprising a CDR1L region of SEQ ID NO: 6, a CDR2L region of SEQ ID NO: 7 and a CDR3L region of SEQ ID NO: 8, wherein the CDRs may comprise any one or more amino acid mutations that does not diminish their activity according to the invention.

Preferably, the CDRs have a sequence identity to their respective SEQ ID NOs of at least 91%, preferably 92%, 93%, 94%, 95%, 96%, 97%, 98%, or 99%.

Preferably, the antibody comprises a VH region comprising a CDR1H region of SEQ ID NO: 3, a CDR2H region of SEQ ID NO: 4 and a CDR3H region of SEQ ID NO: 5, and a VL region comprising a CDR1L region of SEQ ID NO: 6, a CDR2L region of SEQ ID NO: 7 and a CDR3L region of SEQ ID NO: 8.

In another embodiment, antibodies or antigen-binding fragments for use according to invention comprise a heavy chain variable (VH) region having at least 86%, 87%, 88%, 89%, 90%, 91%, 92%, 93%, 94%, 95%, 96%, 97%, 98%, 99%, or 100% sequence identity to an amino acid sequence of SEQ ID NO: 1.

In certain embodiments, a VH sequence having at least 60%, 70%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, whereby the antibody retains the ability to bind specifically to the respective antigen. Preferably, the heavy chain variable region (VH) sequence is SEQ ID NO: 1.

The present invention also relates to an antibody that comprises a light chain variable (VL) region having at least 86%, 87%, 88%, 89%, 90%, 91%, 92%, 93%, 94%, 95%, 96%, 97%, 98%, 99%, or 100% sequence identity to an amino acid sequence of SEQ ID NO: 2.

In certain embodiments, a VL sequence having at least 60%, 70%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, whereby the antibody retains the ability to bind specifically according to the invention to the respective antigen. Preferably, the light chain variable region (VL) sequence is SEQ ID NO: 2.

In certain embodiments, 1 to 10 amino acids have been substituted, inserted and/or deleted in said VL sequences. In other embodiments, 1 to 10 amino acids have been substituted, inserted and/or deleted in said VH sequences. In certain embodiments, 1 to 10 amino acids have been substituted, inserted and/or deleted in each of said VH or VL sequences. Said substitutions, insertions, or deletions may occur in regions outside the CDRs (i.e., in the FRs).

In preferred embodiments, the antibodies are humanized IgG1LALA antibodies, in particular humanized IgG1-type antibodies, having at least two alanine amino acids at positions 234 and 235 of the human Fc1 region. Thus, according to a preferred embodiment, a IgG1LALA comprises the mutation L234A and L235A of the human Fc1 region. In another embodiment, the antibodies comprise at least amino acid substitutions at S228P and L235E of the human IgG4 Fc region.

Further preferred is that the antibodies are recombinant molecules.

The agonistic monoclonal antibody for use according to the invention, or an antigen-binding fragment thereof, is capable of binding to the human CD40 receptor and inducing CD40 signaling independent of Fcγ mediated CD40 receptor crosslinking. Furthermore, it may exhibit reduced or depleted signaling capacity through the human Fcγ receptor when compared to the wildtype IgG Fcγ receptor signaling or to Fcγ signaling of antibodies of prior art.

In certain embodiments, the agonistic monoclonal CD40 antibodies for use according to the invention, or antigen-binding fragments thereof, exhibit a reduced or depleted affinity to human Fcγ receptors compared to the wildtype IgG Fcy. According to a preferred embodiment, the antibodies do not bind to Fcγ receptors-correspondingly the antibodies do not trigger Fcγ mediated CD40 receptor crosslinking.

The antibody for use according to the invention shows rapid binding to rhesus CD40 expressing immune cells after administration and upregulation of costimulatory and homing markers (e.g. CD80, CCR7). Pharmacokinetics analyses show that the antibody or antibody fragment of the present invention persists in the circulation for around a week. Systemic cytokine secretion (e.g. TNF, IL-12) is low. Only upon administration of higher doses, transient increase of IFNγ and IL-6 is observed. The antibody induces a transient drop in platelets and lymphocytes and increase in granulocytes in the periphery, indicative for immune activation. Furthermore, an adjuvant effect to enhance antigen-specific immunity is observed. A dosing in the above range of 0.1 to 1 mg/kg (intravenous or subcutaneous administration) induces comparable and long-term robust cellular and cytokine systemic immune responses. This demonstrates that canonical signaling with the antibody induces a highly potent CD40 activation.

In sum, canonical activation of CD40 with the above defined antibody or fragments thereof induces a relevant systemic immune response in NHPs without toxic effects. The agonistic anti-CD40 antibody of the present invention (MAB273) shows strong activation profile of antigen presenting cells and enhancement of antigen-specific T cell responses and may thus represent a unique adjuvant candidate for inducing cytotoxic T cell immunity.

Further features of the antibodies for use according to the invention are described in WO2019/057792, the disclosure of which is incorporated herein by reference.

According to the present invention, the antibodies or fragments thereof are provided for administration to a patient in a dose of about 0.1 to 1 mg per kg body weight of the patient. Preferably, the dose is less than 1 mg, for example less than 0.9 mg/kg, or less than 0.8 mg/kg. In preferred embodiments, the dose is in a range of about 0.2 to 0.7 mg/kg, 0.3 to 0.6 mg/kg or 0.4 to 0.5 mg/kg.

Due to the favorable properties of the above antibodies, they are capable for use as an immune modulatory agent, in particular in the treatment or prevention of infectious diseases and/or cancer.

In one aspect, the antibodies or fragments thereof are for use in the prevention or treatment of infectious diseases. Infectious diseases include viral infections, (e.g. RNA viruses, DNA viruses, human immunodeficiency virus (HIV), hepatitis A, B, and C virus, herpes simplex virus (HSV), cytomegalovirus (CMV), Epstein-Barr virus (EBV), human papilloma virus (HPV)), parasitic infections (e.g. protozoan and metazoan pathogens such as Plasmodia species, Leishmania species, Schistosoma species, Trypanosoma species), bacterial infections (e.g. Mycobacteria, in particular, M. tuberculosis, Salmonella, Streptococci, E. coli, Staphylococci), fungal infections (e.g. Candida species, Aspergillus species, Pneumocystis carinii), and prions.

The above defined antibodies or fragments thereof are capable to support the immune response in a patient affected by an infectious disease. Moreover, the antibodies or fragments thereof are capable to enhance the immunological response to an immunogen administered to a patient. For example, the antibody or fragment thereof may therefore be administered in or in addition to a vaccine containing an immunogen intended to elicit an immune response in the patient to protect against an infectious disease. Optionally, the antibody or fragment thereof may be combined with further adjuvants and excipients for this purpose.

It will be further appreciated that the patient may also receive one or more further treatments, for example, pharmaceutical agents such as immunotherapeutics. Immunotherapy can for example include immune checkpoint inhibition, and one or more immune checkpoint inhibitors may be used such as anti-TIGIT, anti-PD-L1, anti-PD-1, anti-CTLA-4, anti-CD137, anti-LAG-3, anti-TIM-3, anti-OX40, and/or anti-GITR.

In another aspect, the antibodies or fragments thereof are for use in the treatment of patients suffering from cancer.

Said cancer can be one or more of the types of cancer selected from the group comprising pancreas cancer, advanced pancreatic carcinoma lung cancer, non-small cell lung (NSCL) cancer, bronchioloalviolar cell lung cancer, bone cancer, pancreatic cancer, skin cancer, cancer of the head or neck, cutaneous or intraocular melanoma, ovarian cancer, rectal cancer, cancer of the anal region, stomach cancer, gastric cancer, colon cancer, breast cancer, kidney cancer, Hodgkin's lymphoma, liver cancer, Gall bladder cancer, bladder cancer, prostate cancer, thyroid cancer, salivary gland cancer, or uterine cancer.

In certain embodiments, the cancer is a solid tumor.

It is also possible in some embodiments, that the cancer is a CD40 expressing cancer. However, this is not necessary for the effective functioning of the antibodies.

It will be further appreciated that the antibody may be used as a sole treatment in a patient or as part of a combination treatment (which further treatment may be a pharmaceutical cytotoxic or cytostatic agent, radiotherapy, targeted therapy and/or surgery).

Thus, the patient may also receive one or more further treatments (in particular for an infectious disease or cancer), for example pharmaceutical agents (such as cytotoxic or cytostatic agents, targeted therapy), radiotherapy and/or surgery.

Thus, in some embodiments, the antibody according to the invention is used in the treatment of cancer in combination with cytotoxic or cytostatic agents, radiotherapy, targeted therapy and/or immunotherapy.

The antibodies of the invention can also be used in the treatment of patients that are insufficiently responding and/or resistant to cytotoxic or cytostatic agents, radiotherapy, targeted therapy and/or immune therapy.

Said radiotherapy may be selected from the group comprising external beam radiation therapy, contact x-ray brachytherapy, brachytherapy, systemic radioisotope therapy or intraoperative radiotherapy.

The cytotoxic or cytostatic anti-cancer agents according to the invention may be from the group comprising taxanes, anthracyclins, alkylating agents, histone deacetylase inhibitors, topoisomerase inhibitors, kinase inhibitors, nucleotide analogs, peptide antibiotics, and platinum-based agents.

Preferably the targeted anti-cancer agents are used in targeted therapy and selected from one of the following, or combinations thereof: anti-EGFR compounds such as cetuximab, gefitinib, erlotinib, lapatinib, panitumumab, anti-HER2 compounds such as trastuzumab, ado-trastuzumab emtansine, pertuzumab, VEGF-targeting compounds such as bevacizumab, Aflibercept and Pegaptanib and tyrosine kinase inhibitors such as Sunitinib, Pazopanib, Axitinib, Vandetanib, Cabozantinib and Regorafinib.

In case the patients are receiving immune therapy, this can be immune checkpoint inhibition and one or more immune checkpoint inhibitors may be used. The one or more immune checkpoint inhibitors may be selected from the group comprising anti-TIGIT, anti-PD-L1, anti-PD-1, anti-CTLA-4, anti-CD137, anti-LAG-3, anti-TIM-3, anti-OX40, and/or anti-GITR.

The antibody can also be used in combination with an antibody that specifically binds to human PD-L1, TIGIT, CTLA-4, LAG-3, TIM-3, CD137, OX40, GITR and/or in combination with the drug Nivolumab, Pembrolizumab, Urelumab, Utomilumab, Atezolizumab, Avelumab, Durvalumab, Tremelimumab, Ipilimumab.

In some embodiments according to the invention, the antibody or fragment thereof is used at a weekly to monthly dosing regimen. The dosage should be such that the dose in the patient does not exceed 1 mg/kg body weight at any time. Preferably, the dose in the patient is always less than 1 mg/kg body weight, for example less than 0.9 mg/kg. To obtain an substantial effect, the dose can be kept above 0.1 mg/kg body weight of the patient over a longer period of time.

In another aspect, the present invention relates to a pharmaceutical composition comprising a pharmaceutically acceptable carrier and a therapeutically effective amount of the antibody or antibody fragment, wherein the pharmaceutical composition is adapted for administration of the antibody or fragment in a dose of 0.1 to 1 mg/kg body weight.

The pharmaceutical composition according to the invention may be adapted for controlled release of the antibody or fragment over a longer period of time, preferably a depot formulation for sustained release of the antibody or fragment.

In a specific aspect, the pharmaceutical composition is a vaccine comprising the antibody or fragment thereof described above, in combination with an immunogen. Such a vaccine can be used, in particular, for the prevention of an infectious disease. In this case, the immunogen is a disease-related antigen capable of inducing an immunological response.

In another aspect, the pharmaceutical composition is for use in the treatment of patients suffering from cancer. Such cancer can be a solid tumor. The cancer can also be selected from the group comprising pancreas cancer, advanced pancreatic carcinoma lung cancer, non-small cell lung (NSCL) cancer, bronchioloalveolar cell lung cancer, bone cancer, pancreatic cancer, skin cancer, cancer of the head or neck, cutaneous or intraocular melanoma, ovarian cancer, rectal cancer, cancer of the anal region, stomach cancer, gastric cancer, colon cancer, breast cancer, kidney cancer, Hodgkin's lymphoma, liver cancer, Gall bladder cancer, bladder cancer, prostate cancer, Thyroid cancer, salivary gland cancer, or uterine cancer.

The composition can also be used in the treatment of cancer in combination with chemotherapy, radiotherapy, targeted therapy and/or immunotherapy. Said immunotherapy can be immune checkpoint inhibition.

The patients treated with said composition may be insufficiently responding and/or resistant to chemotherapy, radiotherapy, targeted therapy and/or immune therapy.

The pharmaceutical composition according to the invention may also be used in the treatment of cancer in combination with one or more cytotoxic, cytostatic or targeted anti-cancer compounds.

It can be used in combination with one or more immune checkpoint inhibitors, wherein said immune checkpoint inhibitors may be selected from the group comprising anti-TIGIT, anti-PD-L1, anti-PD-1, anti-CTLA-4, anti-CD137, anti-LAG-3, anti-TIM-3, anti-OX40, and/or anti-GITR.

The composition can also be used in combination with an antibody that specifically binds to human PD-L1, TIGIT, CTLA-4, LAG-3, TIM-3, CD137, OX40, GITR and/or in combination with the drug Nivolumab, Pembrolizumab, Urelumab, Utomilumab, Atezolizumab, Avelumab, Durvalumab, Tremelimumab, Ipilimumab.

It can also be used at a weekly to monthly dosing regimen.

In another aspect, the present invention also relates to methods of treatment, comprising the administration of an effective amount of the antibody to individuals in need of, wherein the dose is in a range of 0.1 to 1 mg/kg body weight. Such individuals may be patients suffering from an infectious disease and/or cancer. Thus, the present invention also relates to methods of treatment of infectious diseases and cancer (e.g. solid tumors).

The step of administering to an individual in need thereof may comprise local administration, for example local administration to a tumor in a patient (for example, intra-tumourally or peri-tumourally). Preferably, administration is subcutaneous or intravenous administration.

For example, the cancer may be selected from the group consisting of: prostate cancer; breast cancer; colorectal cancer; pancreatic cancer; ovarian cancer; lung cancer; cervical cancer; rhabdomyosarcoma; neuroblastoma; multiple myeloma; leukemia, acute lymphoblastic leukemia, melanoma, bladder cancer and glioblastoma.

It will be further appreciated that the methods of treatment according to the invention may comprise the sole administration of the antibody-based agents of the invention to a patient or as part of a combination treatment (which further treatment may be an immunotherapeutic, a pharmaceutical cytotoxic or cytostatic agent, radiotherapy, targeted therapy and/or surgery.

In fact, all features and favorable properties of the antibodies of the invention as detailed above, are also reflected and comprised in the methods of treatment and uses of the antibodies according to the invention.

### EXAMPLES

The following examples are used in conjunction with the figures and tables to illustrate the invention.

### Materials and Methods

### Animal

This study was approved by the Local Ethical Committee on Animal Experiments. Six male (toxicity study)/three male and three female (immunogenicity/biodistribution study) Indian-origin rhesus macaques were housed at Astrid Fagraeus Laboratory, Karolinska Institutet. All procedures were performed according to the guidelines of the Association for Assessment and Accreditation of Laboratory Animal Care.

### Immunizations and Sample Collection [1-4]

The toxicity study was staggered, animals were divided into three groups. The first two animals received intravenous (i.v.) administration of 1 mg/kg anti-CD40 mAb (MAB273, Icano MAB GmbH), the subsequent two animals received i.v. administration of 0.1 mg/kg MAB273, and the last two animals received subcutaneous (s.c.) administration of 0.1 mg/kg MAB273. For i.v. administration, an intravenous infusion in the saphenous vein with the antibody was performed, the total volume was 25 ml and was distribute stepwise as every 5 minutes giving about 4 ml for 1 minute to give 25 ml for 30 minutes. For s.c. administration, a single 0.5 ml subcutaneous injection in the skin above the left quad muscle with antibody was performed. Blood draws were performed at pre-dose, 30 min, four hours, 24 hours, 48 hours, 72 hours, one week, two weeks, three weeks, and four weeks post MAB273 administration in 4 mL heparin tubes. For immunogenicity study, the priming immunizations was performed at day 0. Six macaques were divided into two groups (n=3). Group one received s.c. administration of 0.1 mg/kg HIV Envelope peptides (GenScript, peptides synthesis service) alone, group two received s.c. administration of 0.1 mg/kg HIV Envelope peptides combined with s.c. administration of 0.1 mg/kg MAB273. The boosting immunization was performed at week 7 with the same dose and administration as prime for each group; the second boosting immunization was performed at week 11, all six macaques received s.c. administration of 1 mg/kg HIV Envelope peptides combined with s.c. administration of 0.1 mg/kg MAB273. Blood draws were performed at day 0, 48 hours after prime, week 1, week 2, week 3, week 7, week 8, week 9, week 11, 48 hours after boost, week 12 and week 13; bronchoalveolar lavage (BAL) was taken at week 9 and week 13. For biodistribution study, the Alexa Fluor 680-conjugated MAB273 were injected by s.c. administration of 0.1 mg/kg, the animals were terminated after 24 hours or 48 hours then necropsies were performed, tissues were thereafter collected and analyzed.

### Safety Clinical Chemistry and Hematology Tests [5, 6]

Hematological analyses of heparinized blood were performed within 8 h after collection on an Exigo Vet instrument (Model H400, Boule Diagnostics AB, Sp\dot{a}nga, Sweden) after QC with use of Boule Vet Con control blood. The parameters analyzed were RBC, HCT, MCV, RDW %, RDWa, HGB, MCH, MCHC, PLT, MPV, WBC, LYM, GRAN, and MONO.

Heparinized plasma samples were analyzed using an ABAXIS Vetscan VS2 3.1.35 Chemistry analyzer (Triolab, Solna, Sweden). The parameters analyzed were TBIL, BUN, BA, ALP, ALB, ALT, CHOL, and GGT on Mammalian Liver Profile rotors (Triolab), which have individual QC controls.

Blood sample processing [2-4, 7]Peripheral blood mononuclear cells (PBMCs) were isolated by Ficoll-Paque (GE Healthcare, Fairfield, CT) density gradient centrifugation of blood samples at 2200 rpm for 25 minutes with no brake or acceleration. PBMCs were washed and maintained in phosphate-buffered saline (PBS).

Samples were stained immediately or frozen using 90% heat-inactivated fetal bovine serum (FBS) and 10% DMSO (Sigma-Aldrich) and stored at −170° C.

### Flow Cytometry for Innate Cell Profiles [2, 3, 7, 8]

Fresh PBMCs were stained with LIVE/DEAD™ Fixable Blue Dead Cell Stain Kit (Invitrogen, L23105), then blocked with FcR Blocking Reagent (Miltenyi Biotec, 130-059-901) according to manufacturer's protocol. Followed by a surface staining panel as below:

| Marker | Color | Clone | Vendor | Cat no. | ul/Test |
|---|---|---|---|---|---|
| CD40 | FITC | 5C3 | BioLegend | 334306 | 5 |
| NKg2a | PE | IM3291U | Beckman | Z199 | 2.5 |
| (CD159a) | | | Coulter | | |
| CD80 | BV421 | L307.4 | BD | 564160 | 2.5 |
| CCR7 | PE-Dazzle | G043H7 | Biolegend | 353236 | 2 |
| | 594 | | | | |
| CD123 | PerCp- | 7G3 | BD | 558714 | 1.25 |
| | Cy5.5 | | | | |
| CD3 | APC-Cy7 | SP34-2 | BD | 557757 | 1.25 |
| CD66 | APC | TET2 | Miltenyi | 130-118- | 1.25 |
| | | | | 539 | |
| CD70 | BV786 | Ki-24 | BD | 565338 | 1.25 |
| HLA-DR | BV650 | L243 | Biolegend | 307650 | 1.25 |
| CD11c | PE-Cy7 | 3.9 | BioLegend | 301608 | 0.625 |
| CD16 | AF700 | 38G | BD | 560713 | 0.625 |
| CD20 | BV605 | 2H7 | BioLegend | 302334 | 0.625 |
| CD14 | BV510 | M5E2 | Biolegend | 301842 | 0.625 |

After staining and washing, PBMCs were resuspended in 1% paraformaldehyde (PFA) and acquired on an LSRFortessa flow cytometer (BD). Data analysis was performed using FlowJo v10.

### B Cell Proliferation Assay [2, 4]

Human PBMCs were labeled with 0.25 μM CellTrace Violet (Invitrogen) at a cell concentration of 1 million/ml for 20 min at 37° C. Labeled human PBMCs were stimulated with 1 μg/ml, 0.1 μg/ml, 0.01 μg/ml, or 0.001 μg/ml of MAB273 or anti-CD40 Ab (clone 5C3, BioLegend, 334306). As controls, cells were stimulated with either 1 μg/ml CpG B (Invivogen), 0,2 μg/ml SEB (Sigma), or left unstimulated media in complete (RPMI1640, 10% FBS, 1% L-glutamine, 1% penicillin/streptomycin) and cultured for 6 days. After culture, cells were washed with PBS and stained with LIVE/DEAD™ Fixable Blue Dead Cell Stain Kit (Invitrogen, L23105), then blocked with FcR Blocking Reagent (Miltenyi Biotec, 130-059-901) according to manufacturer's protocol. Followed by a surface staining panel as below:

| Marker | Color | Clone | Vendor | Cat no. | ul/Test |
|---|---|---|---|---|---|
| CD19 | ECD | J3-119 | Beckman | A07770 | 4 |
| CD3 | APC-Cy7 | SP34-2 | BD | 557757 | 1.25 |
| CD20 | BV605 | 2H7 | Biolegend | 302334 | 0.625 |

After staining and washing, PBMCs were resuspended in 1% paraformaldehyde (PFA) and acquired on an LSRFortessa flow cytometer (BD). Data analysis was performed using FlowJo v10.

### T Cell Stimulation [2-4, 7]

Fresh PBMCs or cells from BAL samples were cultured at 1 million cells/ml in 0,2% DMSO+CD107a staining antibody+R10 (unstim), 2 ug/mL HIV Envelope peptides+CD107a staining antibody+R10 or 1 ug/mL SEB+CD107a staining antibody+R10 overnight at 37° C. 6 hours before staining, 10 ug/ml Brefeldin A (BFA; Invitrogen) and 10 ug/ml monensin (Invitrogen) were added to the cultures. After culture, cells were washed with PBS and stained with LIVE/DEAD™ Fixable Blue Dead Cell Stain Kit (Invitrogen, L23105). Followed by a surface staining panel as below:

| Marker | Color | Clone | Vendor | Cat no. | ul/Test |
|---|---|---|---|---|---|
| OX40 | BV510 | L106 | BD | 745040 | 2.5 |
| CCR7 | BV786 | G043H7 | Biolegend | 353230 | 2 |
| CD103 | FITC | 2G5 | Beckman | B49222 | 2 |
| CD8a | BV711 | RPA-T8 | Biolegend | 301044 | 1.25 |
| CD4 | PE-Cy55 | S3.5 | Invitrogen | MHCD0418 | 1.25 |
| CD45RA | BV650 | 5H9 | BD | 740608 | 0.20 |

After staining and washing, cells were permeabilized using Cytofix/Cytoperm kit (BD Biosciences) and stained intracellularly as the panel below:

| Marker | Color | Clone | Vendor | Cat no. | ul/Test |
|---|---|---|---|---|---|
| 4-1BB | APC | 4B4-1 | BD | 550890 | 5 |
| IL-2 | PE | MQ1-17H12 | BD | 559334 | 2 |
| CD69 | ECD | TP1.55.3 | Beckman | 6607110 | 1.5 |
| CD3 | APC-Cy7 | SP34.2 | BD | 557757 | 0.5 |
| IFNg | AF700 | B27 | Biolegend | 506516 | 0.5 |

Cells were washed after staining then resuspended in 1% paraformaldehyde (PFA) and acquired on an LSRFortessa flow cytometer (BD). Data analysis was performed using FlowJo v10.

### Flow Cytometry of Tracking Experiments [2, 4]

Fresh cells from different collected tissues were stained with LIVE/DEAD™ Fixable Blue Dead Cell Stain Kit (Invitrogen, L23105), then blocked with FcR Blocking Reagent (Miltenyi Biotec, 130-059-901) according to manufacturer's protocol. Followed by a surface staining panel as below:

| Marker | Color | Clone | Vendor | Cat no. | ul/Test |
|---|---|---|---|---|---|
| CD1a | PE | SK9 | BD | 333167 | 5 |
| CD209 | PerCP- | DCN46 | BD | 558263 | 5 |
| | Cy55 | | | | |
| CD40 | FITC | 5C3 | BioLegend | 334306 | 5 |
| CD11c | PE-Cy7 | 3.9 | Biolegend | 301608 | 3 |
| CD14 | BV711 | M5E2 | Biolegend | 301838 | 2.5 |
| CD66abce | APC | TET2 | Miltenyi | 130-118- | 2 |
| | | | | 539 | |
| CD45 | BV605 | D058- | BD | 564098 | 2 |
| | | 1283 | | | |
| CCR7 | PE-Dazzle | G043H7 | Biolegend | 353236 | 2 |
| | 594 | | | | |
| CD3 | APC-Cy7 | SP34-2 | BD | 557757 | 2 |
| CD8 | APC-Cy7 | RPA-T8 | BD | 557760 | 2 |
| CD20 | APC-Cy7 | L27 | BD | 335829 | 2 |
| HLA-DR | PE-Cy55 | Tu36 | Life Tech | MHLDR18 | 1 |
| CD123 | BV510 | 6H6 | Biolegend | 306022 | 1 |
| CD80 | BV650 | L307.4 | BD | 564158 | 0.5 |
| CD16 | BV421 | 3G8 | Biolegend | 302038 | 0.5 |

After staining and washing, cells were resuspended in 1% paraformaldehyde (PFA) and acquired on an LSRFortessa flow cytometer (BD). Data analysis was performed using FlowJo v10.

### ELISA Assay for MAB273 Pharmacokinetics Analysis [5, 9]

The concentration of MAB273 in plasma was measured by a custom in-house ELISA assay. Greiner-Bio One 96 well half-area ELISA plates (VWR, 738-0032) were coated overnight at 4° C. with CD40 protein (ThermoFisher, A42565) in fresh PBS. The plates were blocked with PBS containing 5% milk for 1 hour at room temperature (RT). Diluted plasma was added to plates and incubated for 2 hours at RT. MAB273 was detected by adding a 1:5,000 dilution of monkey cross-adsorbed polyclonal goat anti-human IgG HRP antibody (Southern Biotech, 2049-05) and the signal was developed by addition of TMB substrate (BioLegend). The addition of an equal volume of 1M H2SO4 stopped the reaction and the optical density (OD) was read at 450 nm and background was read at 550 nm. The plates were washed 3 times between each incubation step using PBS supplemented with 0.05% Tween 20.

### ELISA Assays for Detection of Plasma Cytokines [2, 3, 7]

Plasma samples from rhesus macaques were evaluated for IFN-gamma, IL-6, TNF and IL-12p70 levels by ELISA kits (EP8RB, BMS223HS, BMS654 and BMS646 from ThermoFisher as well as 3460-1H-6 and 3512M-1H-6 from Mabtech). Assays were performed according to manufacturer's protocol.

### FIGURE LEGEND

FIG. 1: (A) Phenotypic identification of CD20+B cells, CD14+classical monocytes (CM), CD11c+myeloid dendritic cells (MDC) and CD123+plasmacytoid dendritic cells (PDC) in rhesus PBMCs by flow cytometry according to the indicated gating strategy. One representative animal is shown. (B) Baseline expression of CD40 on different rhesus immune cells. Mean fluorescence intensity (MFI) of CD40 is shown. n=4 animals.
First, MAB273 was tested in vitro on rhesus macaque PBMCs to see if the antibody is cross-reactive and the rhesus macaque model could be used to study this antibody in vivo. The flow cytometric gating strategy is indicated as (FIG. 1A).
B cells were found to have the highest baseline expression of CD40, followed by plasmacytoid dendritic cells (PDC) and myeloid dendritic cells (MDC), while classical monocytes (CM) have a relatively low CD40 baseline (FIG. 1B).

FIG. 2: Rhesus PBMCs were stimulated with MAB273 (1, 10, 20 μg/ml), isotype control Ab (1, 10, 20 μg/ml), or TLR7/8L (5 μg/ml) for 24 hours. Surface expression levels of cell activation markers (CD86, CD70), lymph node homing marker (CCR7) and CD40 on MDCs and B cells were evaluated by flow cytometry. Compiled data is from four independent experiments. MFI values are shown, n=4 animals. Two-tailed paired t test was used for statistical analysis.
Then, phenotypic differentiation of the cells after stimulation with MAB273 for 24 hrs was analysed in vitro. We gated on different sub-populations of cells and especially focused on cells that express CD40 (FIG. 1). This showed that:
   MAB273 activates MDCs and B cells to up-regulate the expression of CD86, CD70 and CCR7 (FIG. 2A). No up-regulation was found by the isotype control antibody.
   In terms of CD40 staining, the data indicate that the CD40 staining antibody is blocked by MAB273 binding and therefore we cannot detect CD40 expression after the cells have been cultured with MAB273.
   No or limited activation of PDCs and CMs were found (FIG. 2B).

FIG. 3: Rhesus PBMCs were stimulated with anti-CD40 Ab (1, 10, 20 μg/ml), isotype control Ab (1, 10, 20 μg/ml), or TLR7/8L (5 μg/ml) for 24 hours. Levels of TNF and IL-12 p70 in supernatants collected from cell culture were measured. Compiled data is from four independent experiments. n=4 animals.
This showed that:
   MAB273 stimulated low levels of TNF secretion both at 1 and 10 μg/ml, but it was even lower at 20 μg/ml likely due to toxicity. A TLR7/8 ligand was used as the positive control. In contrast, the isotype control antibody did not induce TNF.
   We did not detect IL-12p70 in the cultures. It is possible that it was produced under the detection level of that ELISA kit.
   In any event, we can conclude that the secretion of large amounts of IL-12p70 were not induced by in vitro stimulation of rhesus PBMCs with MAB273.

FIG. 4: (A) Gating strategy. (B) Human PBMCs were labeled with 0.25 μM CellTrace Violet for 20 min at 37° C., then stimulated with MAB273 (1, 0.1, 0.01, 0.001 μg/ml), anti-CD40 Ab (1, 0.1, 0.01, 0.001 μg/ml), CpG B (1 μg/ml) or SEB (0.2 μg/ml) for 6 days. The status of B cell proliferation was evaluated by flow cytometry.
A B cell proliferation assay after six days of culture with MAB273 was also performed. This showed that:
   detectable B cell proliferation could be observed in PBMCs treated with different concentrations of MAB273, especially in the 0.1 μg/ml group (FIG. 4). However, no proliferation was observed in the anti-CD40 Ab group. The CpG group as a positive control showed strong proliferation as expected.
FIG. 5: Fresh blood samples (350 μl) were sent to Adlego Biomedical AB (based at Karolinska Institutet Science Park) for clinical chemistry testing. n=2 animals per group.
After the in vitro testing we tested three different conditions of MAB273 in vivo by dividing six macaques and into three groups. We staggered the study, starting with 1 mg/kg administered i.v. and a 10-fold lower dose for the second group and also for the third group but administered s.c. Blood draws were done frequently after administration and the animals were followed for 4 weeks. We performed clinical chemistry testing, which is a series of tests of liver and kidney function.
This showed that:
   For the 1 mg/kg i.v. group, several parameters were elevated above the healthy reference range. This group also showed some side effects in terms of loss of appetite, vomiting and change of behavior.
   The reduced dose at 0.1 mg/kg i.v. did not induce marked side effects.
   The dose at 0.1 mg/kg with s.c. administration did not induce marked side effects.
   For 0.1 mg/kg group (both i.v. and s.c.), almost all parameters stayed within the normal range (FIG. 5).

FIG. 6: Fresh blood samples (350 μl) were sent to Adlego Biomedical AB (based at Karolinska Institutet Science Park) for complete blood counts (CBC). n=2 animals per group.
By complete blood counts, there were notable fluctuations of cell numbers following administration of MAB273.
This showed that:
   A rapid decline in platelets and a rapid increase in granulocytes was observed already at half hour to four hours post-administration in all groups (FIG. 6).
   Cell number fluctuations were dose dependent, with a notably extended effect in the 1 mg/kg i.v. group.

FIG. 7: The concentration of MAB273 in plasma after administration was measured by a custom in-house ELISA assay. ULOD=upper limit of detection (67967.1 ng/ml), LLOD=lower limit of detection (0.047 ng/ml), LLOQ=lower limit of quantitation (0.261 ng/mL). n=2 animals per group.
ELISA assays were used to measure the concentration of MAB273 in plasma after administration.
This showed that:
   MAB273 was well detectable already after 30 min after administration especially in the i.v. groups (FIG. 7).
   For the high dose group, the peak of MAB273 concentration appeared around 30 minutes to 4 hours, then began to decline gradually until undetectable at day 14.
   For the low dose i.v. group, the highest concentration was detected at 30 minutes and continually decreased until undetectable at day 7.
   For the low dose s.c. group, the peak of MAB273 concentration appeared later (around day 3) and remained at this level longer until undetectable at day 7-14. The highest concentration was relatively low compared with the other two groups.

FIG. 8: (A) Gating strategy. (B) Cell frequencies from rhesus PBMCs were evaluated by flow cytometry. n=2 animals per group.
To further break down the fluctuations of immune cell numbers on the subset level and to analyze their activation status, a 14-parameter flow cytometry panel was used to follow PBMC samples longitudinally (FIG. 8A). Analyzed cell subset frequencies were normalized to the complete blood count (CBC) data (FIG. 6).
This showed that:
   Cell fluctuations followed a similar trend to the CBC data with a rapid increase in neutrophils and a transient decline in both B cells and MDCs (FIG. 8B).
   The observed kinetics can be interpreted as a re-distribution of immune cells from circulation to tissues and then a re-population of new immune cells from the bone marrow.

FIG. 9: Cell surface expression of CD40 on rhesus PBMCs was evaluated by flow cytometry. Mean fluorescence intensity (MFI) of CD40 is shown. n=2 animals per group.
Targeting of cell surface CD40 by MAB273 was evaluated by quantifying the loss of binding of a competing fluorescent antibody by flow cytometry (as described in FIG. 2).
This showed that
   MAB273 rapidly and effectively sequestered CD40 on B cells and MDCs (FIG. 9).
   The kinetics of CD40 detection followed the pharmacokinetics of MAB273 in plasma (FIG. 7).
   The return of detectable CD40 expression may be due to the appearance of new cells from the bone marrow as well as the half-life of the antibody in vivo.

FIG. 10: Cell surface expression of CD80, a co-stimulatory marker, on rhesus PBMCs was evaluated by flow cytometry. Mean fluorescence intensity (MFI) of CD80 is shown. n=2 animals per group.
Activation by phenotyping was also analyzed by flow cytometry.
This showed that:
   There was an increase in CD80 expression, a co-stimulatory marker, directly after MAB273 administration followed by a decrease in B cells, especially in i.v. administration groups (FIG. 10).
   A rapid phenotypic maturation of APCs may be followed by that the cells leave the circulation and a re-population of new immature APCs takes place.

FIG. 11: CCR7, a lymph node homing marker, on rhesus PBMCs were evaluated by flow cytometry. Mean fluorescence intensity (MFI) of CCR7 is shown. n=2 animals per group.
Similar to CD80 expression, we found that
   expression of CCR7, a lymph node homing marker, also rapidly increased and later decreased on B cells, especially in i.v. administration groups (FIG. 11).
   The reason probably is the same as for CD80.

FIG. 12: Levels of plasma cytokines (IFN-gamma and IL-6) were measured according to manufacturer's protocol using ELISA kits. n=2 animals per group.
Systemic levels of proinflammatory cytokines in plasma were assessed by ELISA. We detected that:
   IFN-γ and IL-6 both increased significantly within the first or second day of high dose administration only (FIG. 12)
   TNF-α and IL-12 p70 were out of detectable range, even when we ran additional high sensitivity ELISA kits (TNF ELISA kits: BMS223HS, BMS654, 3512M-1H-6. IL-12 p70 ELISA kit: BMS646).
This showed that:
   The lack of systemic cytokine release with the lower dose groups is in line with the better safety profile observed.

FIG. 13: Ag-specific T cells in PBMCs (A) and BAL (B) over time. Compiled data are background subtracted. The prime and boost immunizations were performed on week 0 and week 7, respectively (3 animals received s.c. administration of 0.1 mg/kg HIV Envelope peptides alone, while another 3 animals received s.c. administration of 0.1 mg/kg HIV Envelope peptides combined with s.c. administration of 0.1 mg/kg MAB273, both in prime and boost).
A second boost immunization was also performed. This time, all 6 animals received s.c. administration of 1 mg/kg HIV Envelope peptides combined with s.c. administration of 0.1 mg/kg MAB273 on week 11. Individual data are shown as dots. n=3 animals per group.
This showed that:
   Envelope-specific CD4 and CD8 T cell responses were induced in both groups but at low levels. A suboptimal dose of the HIV 1-envelope peptides 0.1 mg/kg may have been used.
   Despite this, overall higher frequencies of envelope-specific CD4 and CD8 T cells were found in the MAB273 group compared to the group that received peptides only.
   The second boost including MAB273 plus higher dose of peptides to all animals showed a boosting effect of T cell responses in animals in both groups.
   In addition to responses in PBMCs, T cell responses were also detectable in BAL.

FIG. 14: Histograms of Alexa Fluor 680 signal from different cell population in different tissues. Control=peripheral blood B cells from the same animal but right before tracking immunization. One representative animal is shown. n=3 animals.
Finally, to assess the biodistribution of MAB273 after administration, three animals received 0.1 mg/kg Alexa Fluor 680-conjugated MAB273 by s.c. administration. The animals were terminated after 24 hours or 48 hours and necropsies were performed to assess tissues for presence of MAB273.
This showed that.
   The Alexa Fluor 680 signal was exclusively detected at the site of injection (L. Skin) and the specific LNs draining the injection site (L. Inguinal, L. Com. Iliac and Paraaortic LNs)
   The Alexa Fluor 680 signal was gradually reduced the more distant the LNs were from the site of injection
   Compared to 24 hr, more MAB273 had reached more distant LNs at 48 hrs.
   Major cell populations that had been targeted by MAB273 in vivo were monocytes, MDCs, neutrophils while T cells showed low targeting as expected.
   No detection of MAB273 on PBMCs was found possibly caused by anti-MAB273 IgG hindering systemic dissemination in these non-naïve animals.

FIG. 15: Sequences (amino acids in one letter code)

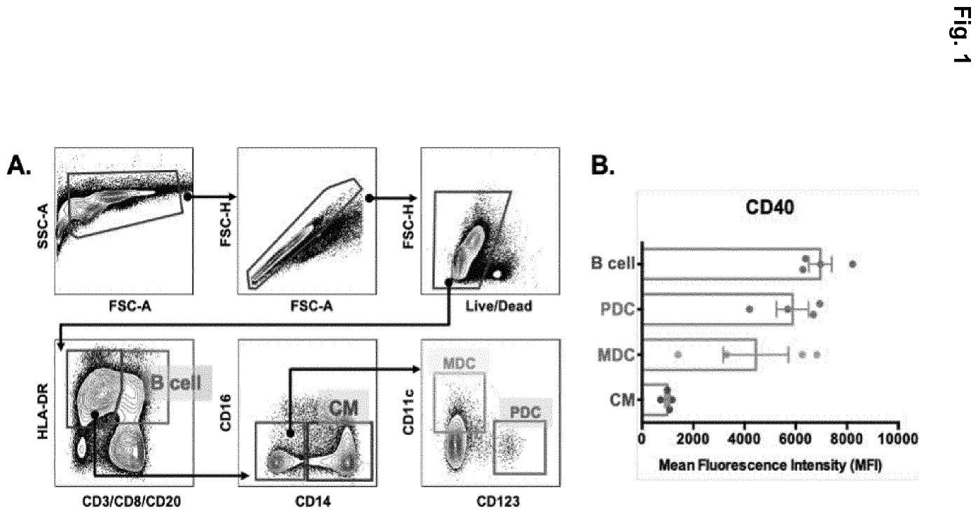

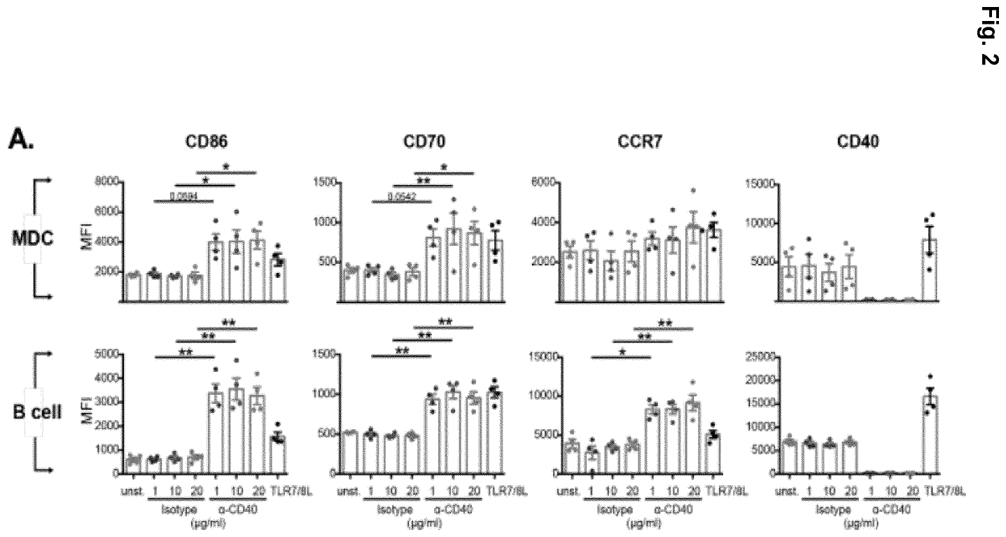

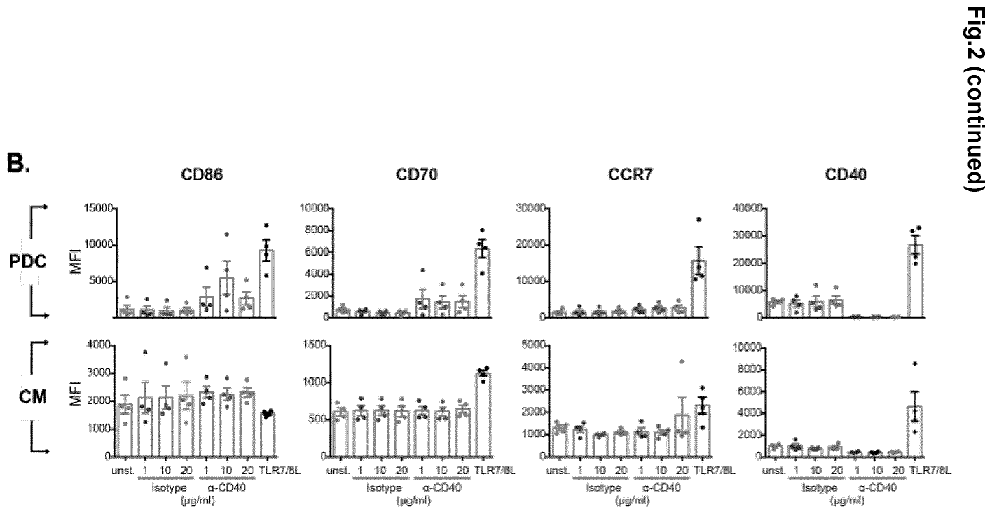

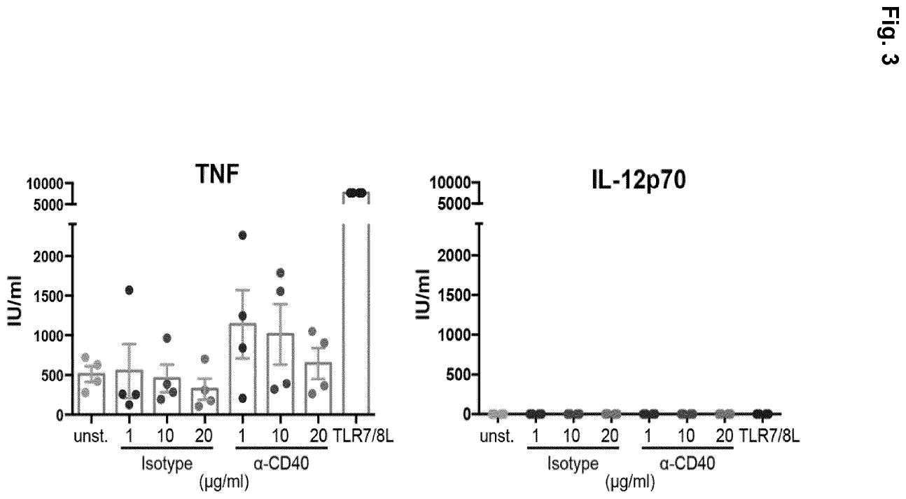

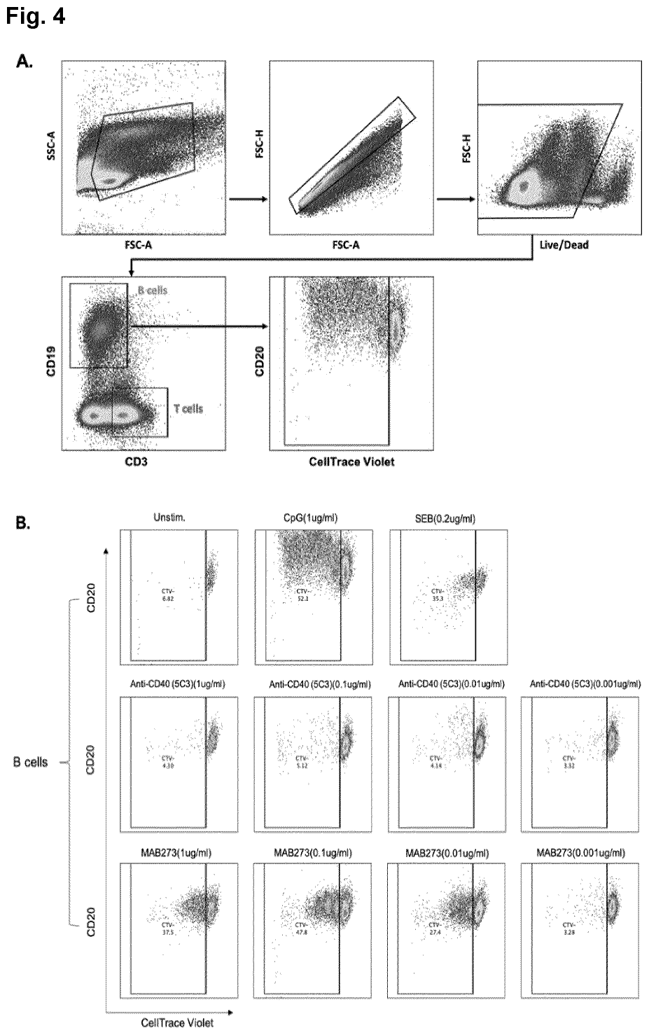

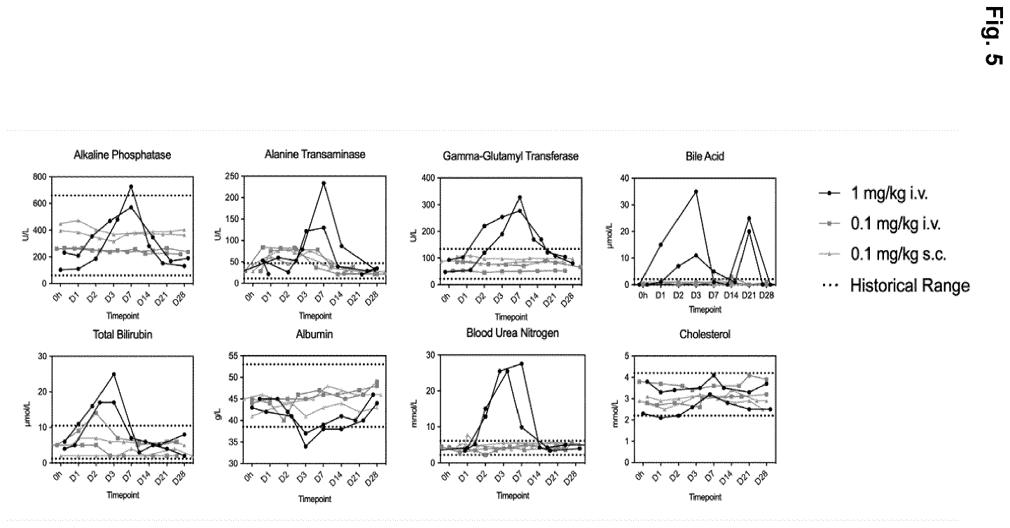

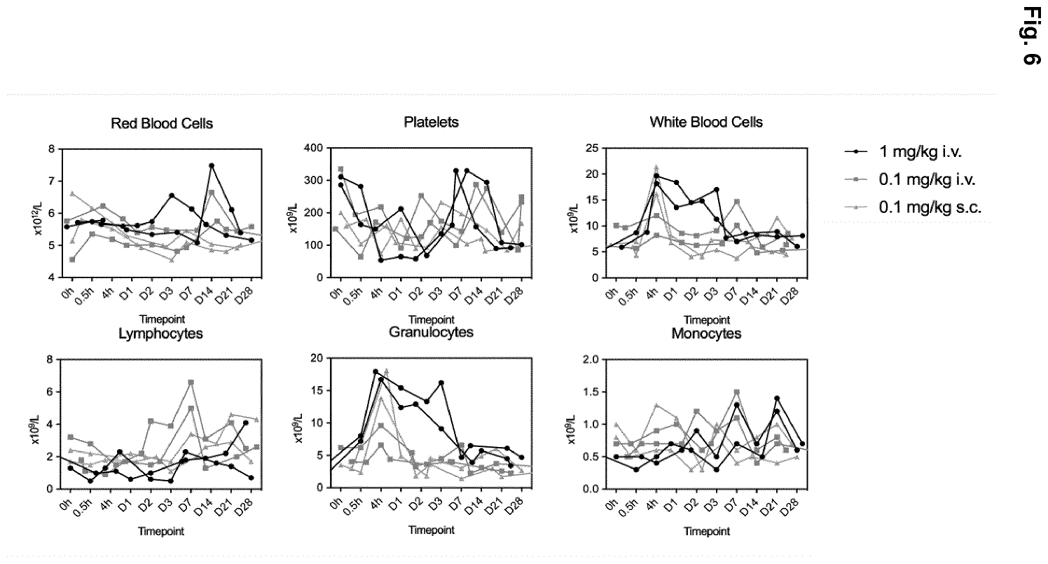

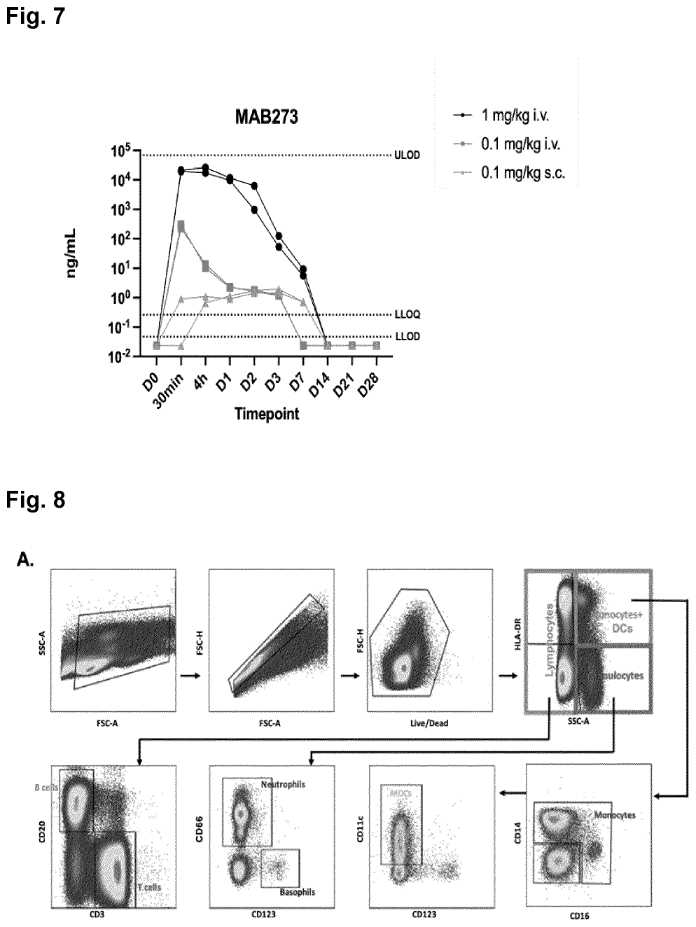

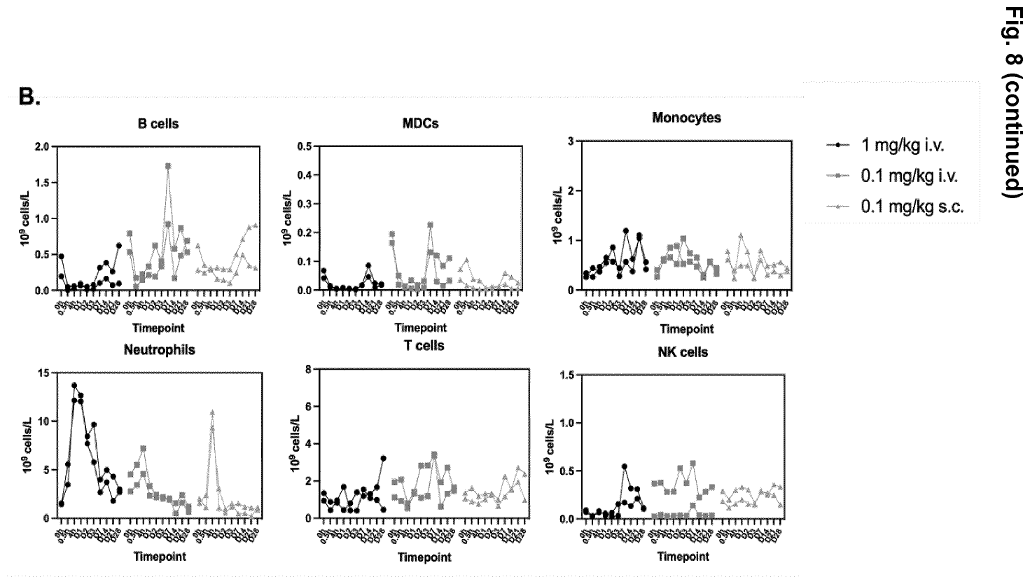

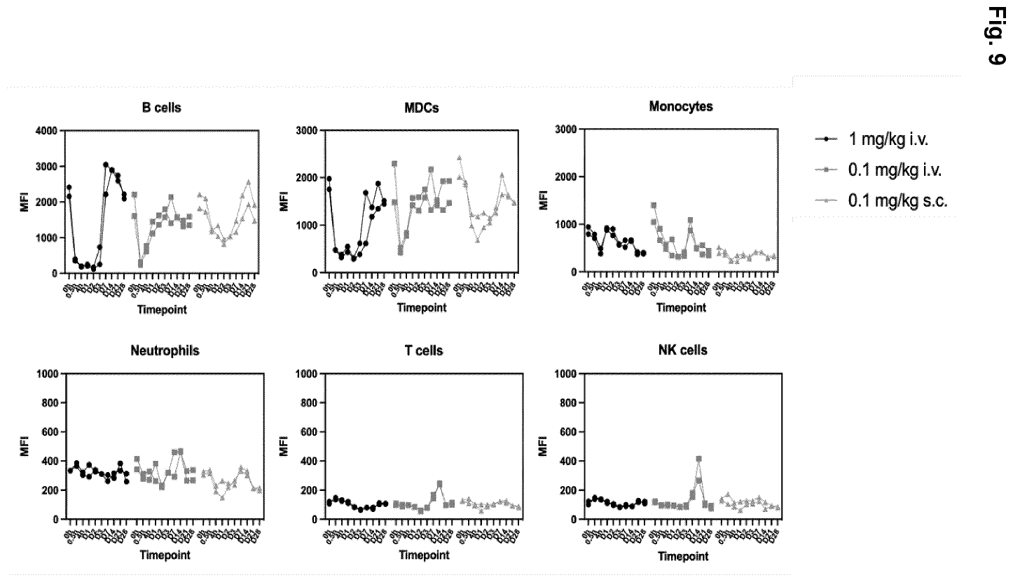

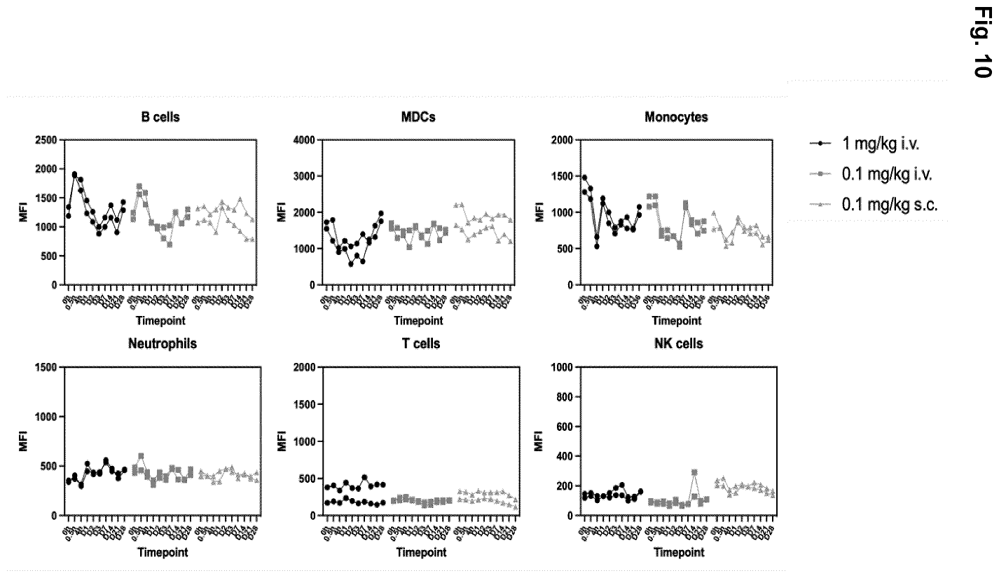

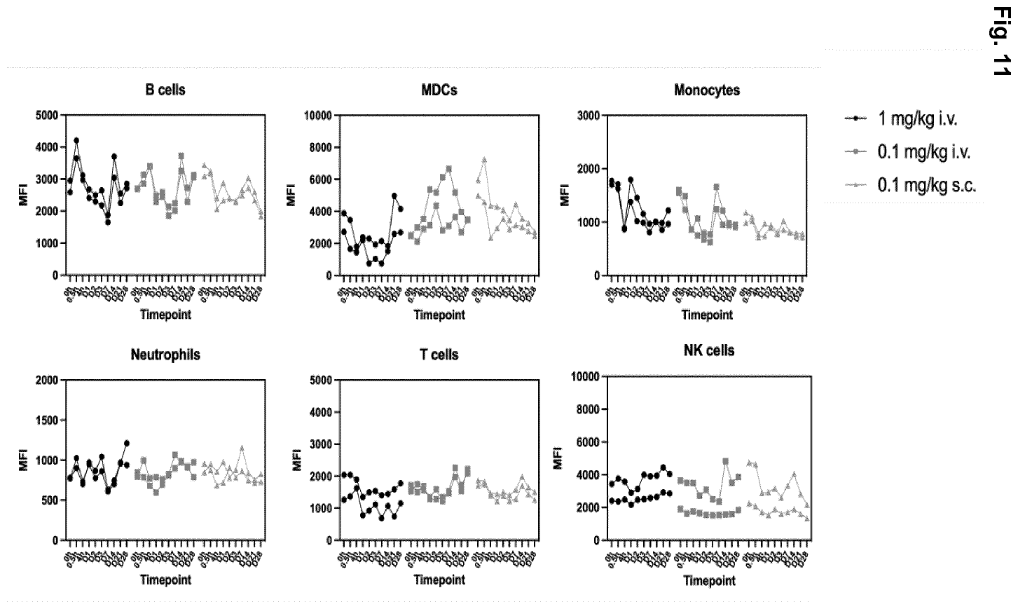

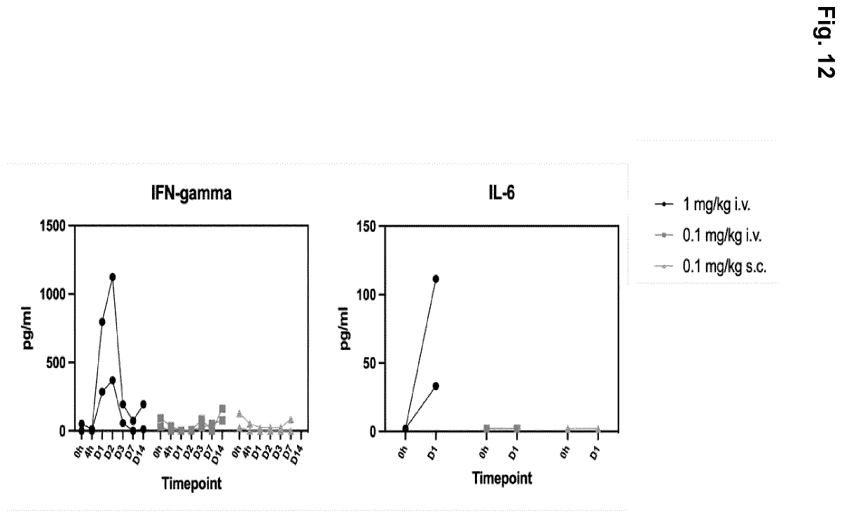

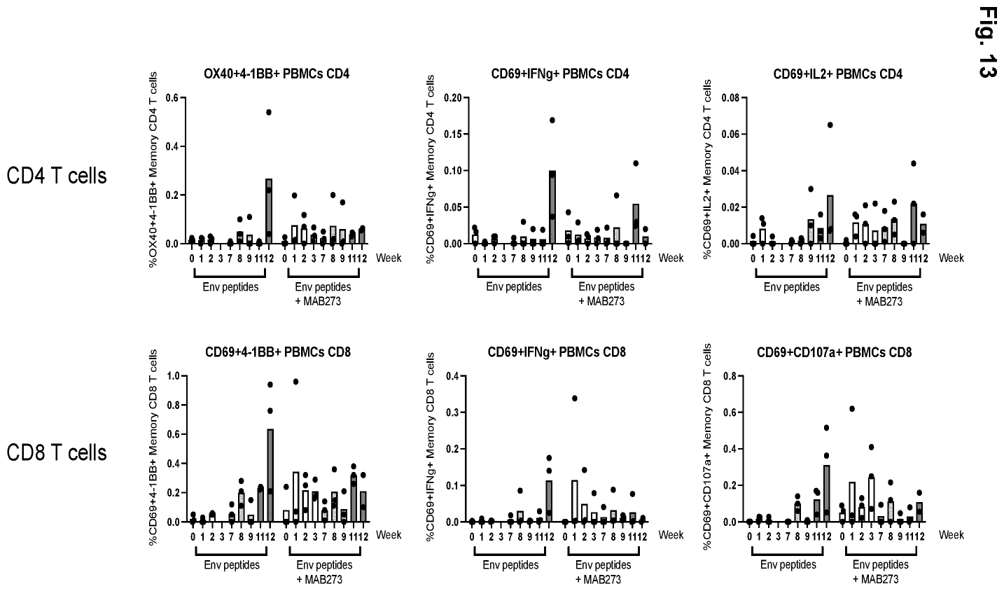

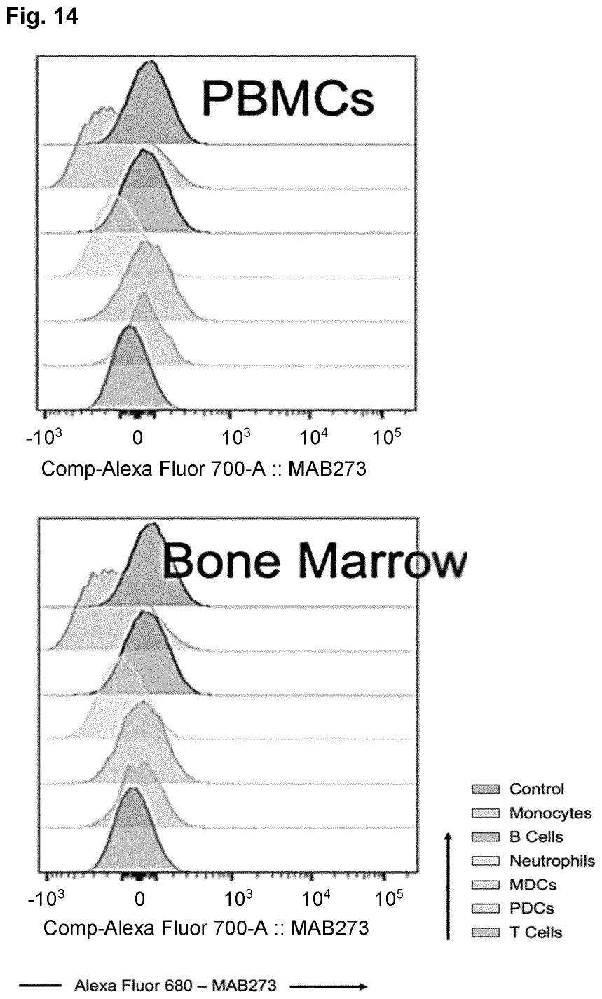

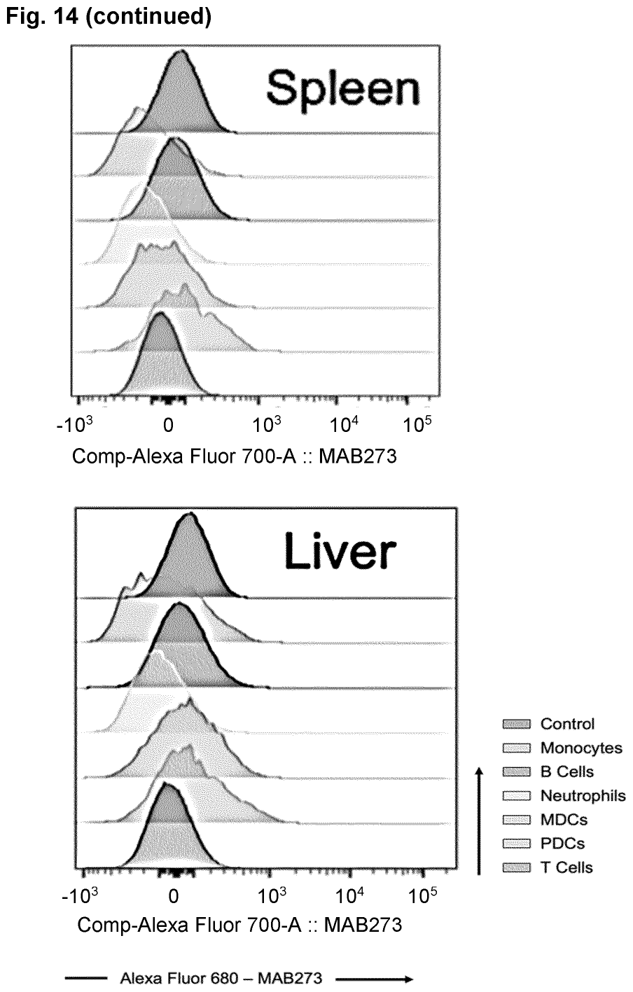

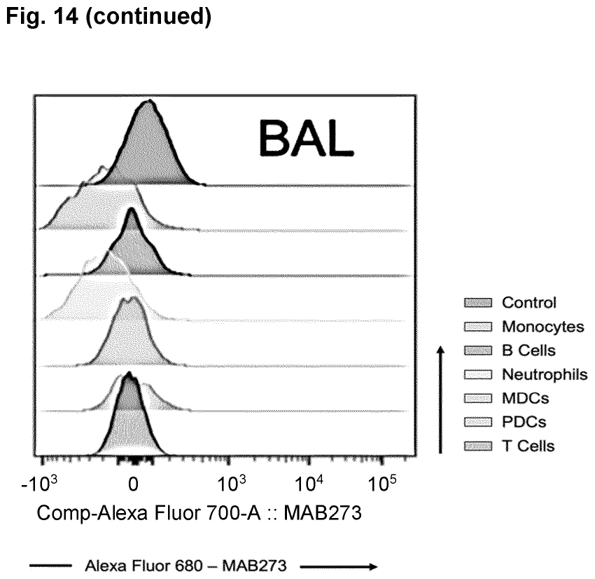

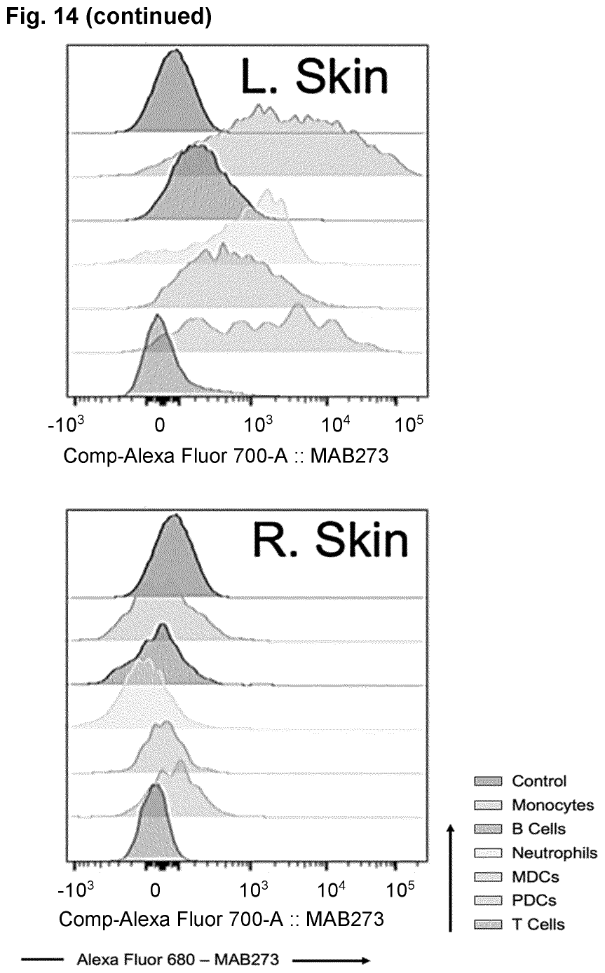

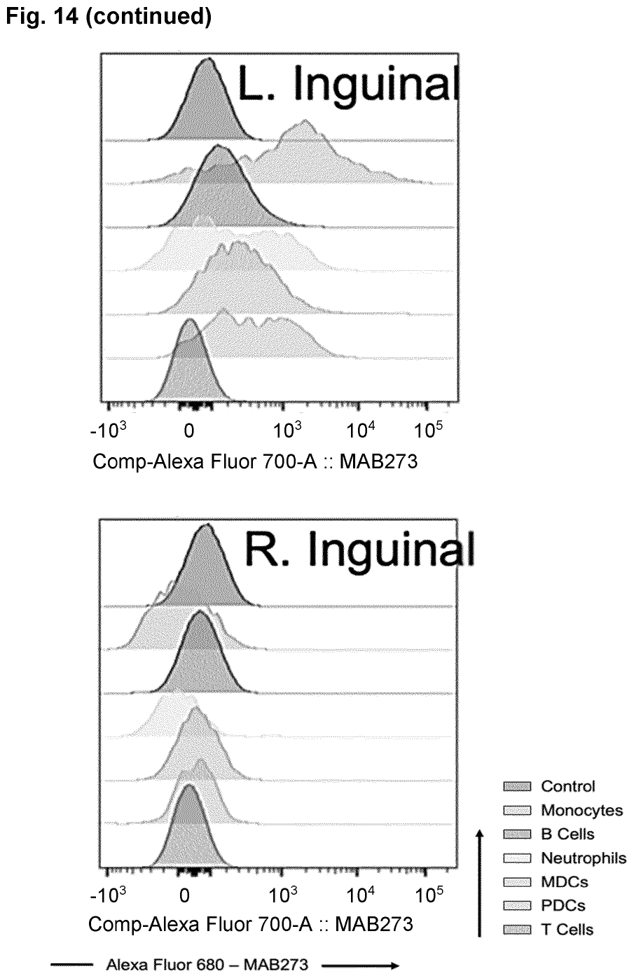

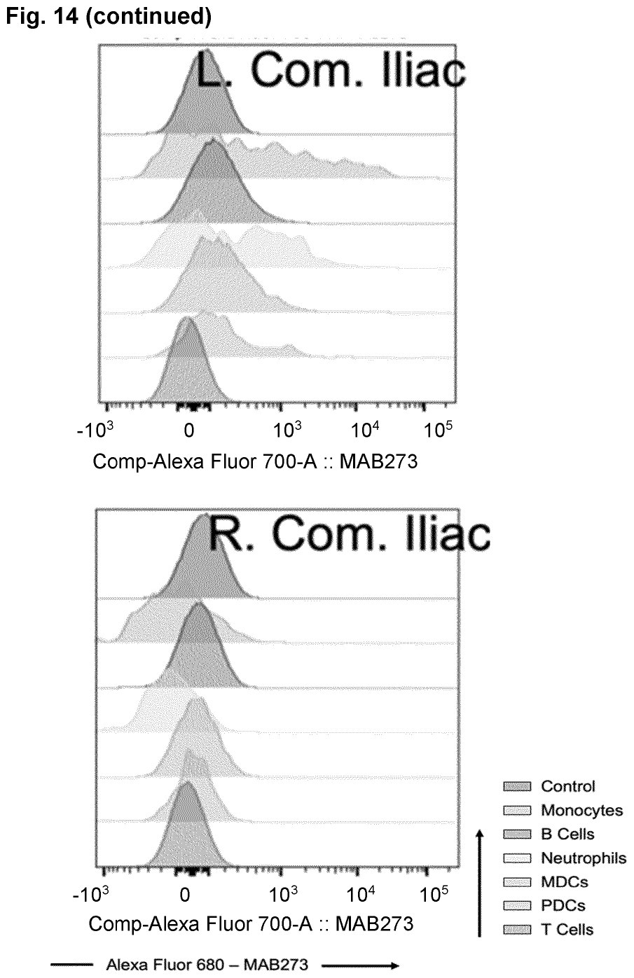

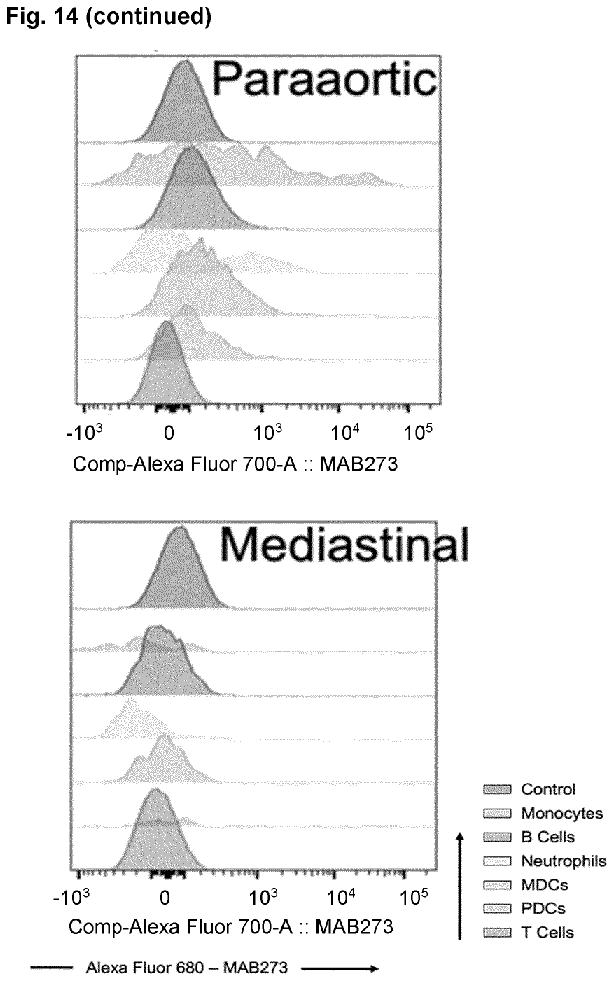

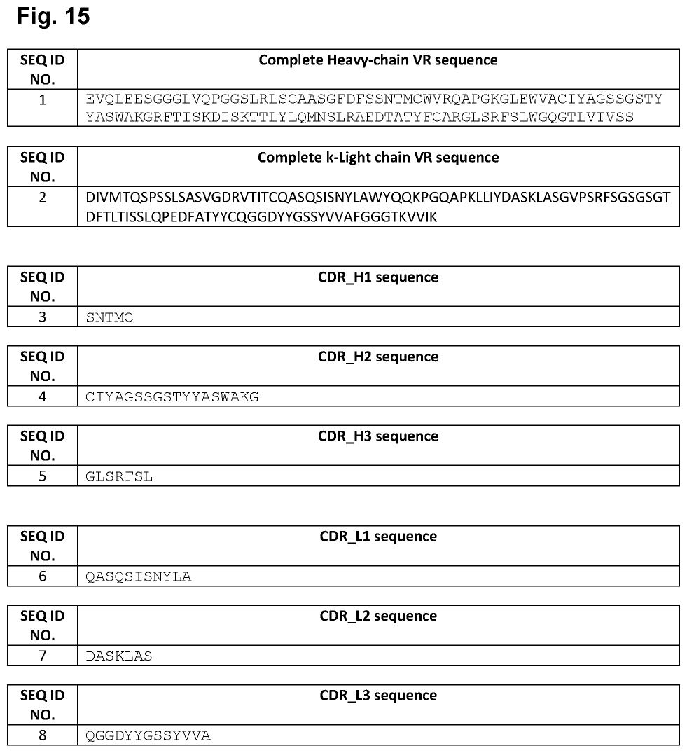

Complete sequences of Variable Regions (VR):
Heavy chain: VH complete: SEQ ID NO: 1
Light chain: VL complete: SEQ ID NO: 2

### Complementary Determining Regions (CDR):

Heavy Chain: CDR-H1: SEQ ID NO: 3
CDR-H2: SEQ ID NO: 4
CDR-H3: SEQ ID NO: 5
Light Chain: CDR-L1: SEQ ID NO: 6
CDR-L2: SEQ ID NO: 7
CDR-L3: SEQ ID NO: 8

### REFERENCES

1. F, L., et al., Efficient Targeting and Activation of Antigen-Presenting Cells In Vivo after Modified mRNA Vaccine Administration in Rhesus Macaques. Molecular therapy: the journal of the American Society of Gene Therapy, 2017. 25 (12).
2. EA, T., et al., Human Anti-CD40 Antibody and Poly IC: LC Adjuvant Combination Induces Potent T Cell Responses in the Lung of Nonhuman Primates. Journal of immunology (Baltimore, Md.: 1950), 2015. 195 (3).
3. EA, T., et al., Monocytes Acquire the Ability to Prime Tissue-Resident T Cells via IL-10-Mediated TGF-β Release. Cell reports, 2019. 28 (5).
4. S, O., et al., Route of Vaccine Administration Alters Antigen Trafficking but Not Innate or Adaptive Immunity. Cell reports, 2020. 30 (12).
5. J, R., et al., Immune modulation with weekly dosing of an agonist CD40 antibody in a phase I study of patients with advanced solid tumors. Cancer biology & therapy, 2010. 10 (10).
6. DA, K., D. R, and R. JV, Toxicity of an Fc-engineered anti-CD40 antibody is abrogated by intratumoral injection and results in durable antitumor immunity. Proceedings of the National Academy of Sciences of the United States of America, 2018. 115 (43).
7. EA, T., et al., TLR-adjuvanted nanoparticle vaccines differentially influence the quality and longevity of responses to malaria antigen Pfs25. JCI insight, 2018. 3 (10).
8. F, L., et al., Dissociation of skeletal muscle for flow cytometric characterization of immune cells in macaques. Journal of immunological methods, 2015. 425.
9. NH, S., et al., Results from an Integrated Safety Analysis of Urelumab, an Agonist Anti-CD137 Monoclonal Antibody. Clinical cancer research: an official journal of the American Association for Cancer Research, 2017. 23 (8).

1. Agonistic monoclonal antibody, or an antigen-binding fragment thereof, that specifically binds to the human CD40 receptor and is capable of inducing CD40 signaling independent of Fcγ mediated CD40 receptor crosslinking, wherein the antibody comprises
a) a VH region comprising a CDR1H region of SEQ ID NO: 3, a CDR2H region of SEQ ID NO: 4 and a CDR3H region of SEQ ID NO: 5, and
b) a VL region comprising a CDR1L region of SEQ ID NO: 6, a CDR2L region of SEQ ID NO: 7 and a CDR3L region of SEQ ID NO: 8,
wherein the CDRs may comprise any one or more amino acid mutations that do not diminish their activity according to the invention,
for medical use, in particular as an immune stimulatory agent, wherein the antibody or fragment thereof is administered in a dose of 0.1 to 1 mg/kg body weight.

2. The antibody or fragment thereof for use according to claim 1, wherein the antibody or fragment thereof is administered in a dose of 0.2 to 0.9 mg/kg body weight, preferably 0.3 to 0.7 mg/kg, or 0.4 to 0.5 mg/kg.

3. The antibody or fragment thereof according to claim 1, wherein the antibody is a humanized IgG1LALA antibody.

4. The antibody or fragment thereof according to claim 3, wherein the antibody comprises at least amino acid substitutions at L234A and L235A of the human IgG1 Fc region or S228P and L235E of the human IgG4 Fc region.

5. The antibody or fragment thereof according to claim 1, wherein the antibody comprises a heavy chain variable (VH) region that is at least 85% identical to a VH region of SEQ ID NO: 1.

6. The antibody or fragment thereof according to claim 1, wherein the antibody comprises a light chain variable (VL) region that is at least 85% identical to a VL region of SEQ ID NO: 2.

7. The antibody or fragment thereof for use according to claim 1, in the prevention or treatment of an infectious disease and/or cancer.

8. The antibody or fragment thereof for use according to claim 7, wherein the cancer is a solid tumor.

9. The antibody or fragment thereof for use according to claim 7, wherein the cancer is selected from the group consisting of pancreas cancer, advanced pancreatic carcinoma lung cancer, non-small cell lung (NSCL) cancer, bronchioloalveolar cell lung cancer, bone cancer, pancreatic cancer, skin cancer, cancer of the head or neck, cutaneous or intraocular melanoma, ovarian cancer, rectal cancer, cancer of the anal region, stomach cancer, gastric cancer, colon cancer, breast cancer, kidney cancer, Hodgkin's lymphoma, liver cancer, Gall bladder cancer, bladder cancer, prostate cancer, thyroid cancer, salivary gland cancer, and uterine cancer.

10. The antibody or fragment thereof for use according to claim 1, wherein the antibody or fragment thereof is used in combination with cytotoxic or cytostatic agents, radiotherapy, targeted therapy, immunotherapy or surgery.

11. (canceled)

12. (canceled)

13. Pharmaceutical composition comprising a pharmaceutically acceptable carrier and a therapeutically effective amount of the antibody or fragment according to claim 1, wherein the pharmaceutical composition is adapted for administration of the antibody or fragment in a dose of 0.1 to 1 mg/kg body weight.

14. The pharmaceutical composition according to claim 13, adapted for controlled release of the antibody or fragment over a longer period of time, preferably a depot formulation for sustained release of the antibody or fragment.

15. The pharmaceutical composition according claim 13, comprising a pharmaceutically acceptable carrier suitable for intravenous or subcutaneous administration.

16. A method for stimulating a patient's immune system, comprising administering an agonistic monoclonal antibody, or an antigen-binding fragment thereof, that specifically binds to the human CD40 receptor and is capable of inducing CD40 signaling independent of Fcγ mediated CD40 receptor crosslinking to said patient, wherein the antibody comprises
a) a VH region comprising a CDR1H region of SEQ ID NO: 3, a CDR2H region of SEQ ID NO: 4 and a CDR3H region of SEQ ID NO: 5, and
b) a VL region comprising a CDR1L region of SEQ ID NO: 6, a CDR2L region of SEQ ID NO: 7 and a CDR3L region of SEQ ID NO: 8,
wherein the CDRs may comprise any one or more amino acid mutations that do not diminish their activity according to the invention, and
wherein the antibody or fragment thereof is administered in a dose of 0.1 to 1 mg/kg body weight.

17. The method according to claim 16, wherein the antibody or fragment thereof is administered in a dose of 0.2 to 0.9 mg/kg body weight, 0.3 to 0.7 mg/kg body weight, or 0.4 to 0.5 mg/kg body weight.

18. The method according to claim 16, wherein said patient is suffering from an infectious disease and/or cancer.

19. The method according to claim 16, wherein the cancer is a solid tumor.

20. The method according to claim 18, wherein the cancer is selected from the group consisting of pancreas cancer, advanced pancreatic carcinoma lung cancer, non-small cell lung (NSCL) cancer, bronchioloalviolar cell lung cancer, bone cancer, pancreatic cancer, skin cancer, cancer of the head or neck, cutaneous or intraocular melanoma, ovarian cancer, rectal cancer, cancer of the anal region, stomach cancer, gastric cancer, colon cancer, breast cancer, kidney cancer, Hodgkin's lymphoma, liver cancer, Gall bladder cancer, bladder cancer, prostate cancer, thyroid cancer, salivary gland cancer, and uterine cancer.

21. The method according to claim 16, wherein the antibody or fragment thereof is administered in combination with at least one immune checkpoint inhibitor, preferably at least one immune checkpoint inhibitor selected from the group consisting of anti-TIGIT, anti-PD-L1, anti-PD-1, anti-CTLA-4, anti-CD137, anti-LAG-3, anti-TIM-3, anti-OX40, and/or anti-GITR.

22. The method according to claim 16, wherein the antibody or fragment thereof is administered as a single dose or as multiple doses.
